(19) United States
(12) Patent Application Publication
(10) Pub. No.: US 2008/0300605 A1
(43) Pub. Date: Dec. 4, 2008

(54) Pedicle protractor tool

(76) Inventor: James A. Rinner, Franksville, WI (US)

Correspondence Address: BAKER & HOSTETLER LLP, WASHINGTON SQUARE, SUITE 1100, 1050 CONNECTICUT AVE. N.W., WASHINGTON, DC, 20036-5304, US

(21) Appl. No.: 11/797,568

(22) Filed: Jun. 1, 2007

(51) Int. Cl. A61B 17/90

(52) U.S. Cl. 606/96

(57) ABSTRACT

A surgical hand tool is provided having a plumb assembly fixed to a dome housing element. The plumb assembly is fixed to rotate freely in two axes of rotation in at least two rotational planes. A tool attachment coupling such as bushing for receiving a surgical instrument such as a drill or tap is fixed to the dome housing. A handle can be fixed to the device for use and orientation by a surgeon or other user. The hand tool can be used as a protractor for orienting surgical instruments such as pedicle screws in at least two angles on two planes for accurately applying said instruments into the pedicles of a vertebra. Generally, the tool of the present invention can be used to accurately orient an instrument relative to a body in at least two angles in two planes.

### FIELD OF THE INVENTION

This invention relates to medical and surgical devices, in particular, to a device for orienting a surgical tool relative to a body.

### BACKGROUND OF THE INVENTION

In orthopedic and spinal surgery, the correct placement of surgical tools and objects, such as a tap, drill, or screw, into various bodies or portions of the anatomy is critical. One such anatomical body demanding extreme precision during placement is the pedicle of the spinal vertebrae. If placement of an object into a pedicle is not done properly, the object could breach the wall of the pedicle, resulting in various injuries, tissue or neurological damage, or other serious complications. There exists a need therefore, to accurately position surgical elements such as screws into an anatomical body such as a pedicle.

There are currently two methods of orientation or insertion of surgical elements into a pedicle. One method is visual, utilizing x-rays or radiological images and then approximating the position by hand. The inaccuracies of this first method are inherently obvious. The second method uses extremely expensive image-guided systems. Despite the two existing methods, several known studies report that procedures such as the placement of pedicle screws have unacceptably high rates of malpositioning of screws, perforations through the pedicle cortex, violations of the pedicle wall, and/or breach of the cortical wall.

In one paper by I. H. Kalfas, “Image-Guided Spinal Navigation: Application to Spinal Metastases,” Neurosurg Focus 11(6), 2001, American Ass'n of Neurological Surgeons (hereinafter “Kalfas”), the shortcomings of the known art in this field are summed up well: “ . . . surgical techniques place a greater demand on the spine surgeon by requiring a precise spatial orientation to that part of the spinal anatomy that is not exposed in the surgical field . . . various reconstruction techniques that require placing bone screws into the pedicles of the thoracic, lumbar, and sacral spine require ‘visualization’ of the unexposed spinal anatomy. Although conventional intraoperative imaging techniques such as fluoroscopy have proven useful, they provide only two-dimensional imaging of a complex 3D structure. Consequently, the surgeon is required to extrapolate the third dimension based on an interpretation of the images and a knowledge of the pertinent anatomy. This so-called ‘dead reckoning’ of the anatomy can result in varying degrees of inaccuracy when placing screws into the unexposed spinal column.” (Kalfas, “Introduction”, para. 2) And in particular with respect to pedicles, Kalfas writes that: “Because of the variations of pedicle anatomy within each patient, however, safe and precise placement of pedicle screws can be difficult. Sub-optimum screw placement can result in varying degrees of neural injury and fixation-related failure. These complications can be minimized if, prior to screw placement, the surgeon is provided with accurate spatial orientation to each pedicle to be instrumented.” (Kalfas, “Pedicle Fixation”, para. 1)

Thus, as enumerated in Kalfas, the pitfalls of image-guided placement of surgical elements are at least two-fold. First, like any other computer-based modality, image-guided navigation is highly dependent on the quality of the information imported into the system. Although properly formatted CT scans need to be obtained and the data correctly transferred to the navigational workstation, the critical step in image guidance is actually the registration process. If the surgeon takes too casual an approach to registration, inaccurate information will be displayed during intraoperative navigation. Second, image guidance is dependent on the correlation of navigational data with the surgeon's own knowledge of the anatomy and the appropriate trajectories through that anatomy. Image-guided navigation is not a replacement for the surgeon's knowledge of anatomy and surgical technique. Rather, it merely serves to help confirm his or her estimation of the nonexposed anatomy by providing imaging data that typically exceeds those yielded by intraoperative fluoroscopy. Thus, image-guided surgery must always be used in conjunction with a knowledge of spinal anatomy. If equipment is not properly registered to correct landmarks the surgeon could be placing the screw at the wrong angle or even in the wrong level. Even the most modern image guided systems come with the warnings noted in Kalfas, and often contain a disclaimer that the system should be used only by qualified medical professionals who have been trained and are experienced in its use, and that the system should be used only as an adjunct for surgical guidance and not as a replacement for the surgeon's knowledge, expertise, or judgment.

The accurate placement of a surgical element therefore depends on knowledge of anatomy, landmarks associated with the anatomy, and extraction of information from images obtained of the anatomy. Once the aforesaid information is assembled and analyzed, a surgical instrument such as a pedicle screw must then be inserted, its orientation and position being determined by the shapes and features such as an angle or plane of the anatomy. Insertion of a drill or screw can involve taking into account multiple planes or angles of orientation of an anatomical body. For a pedicle, this involves at least two angles in at least two planes.

The angles and planes used with respect to a pedicle involve reference planes commonly understood in human anatomy, such as: (i) the “median” plane, being an imaginary vertical plane passing lengthwise through the midline of the body, anterior to posterior, dividing the body into equal left and right halves (a “sagittal” or “lateral” plane is any plane parallel to the median plane, dividing the body into unequal left and right halves, such that the median plane is also referred to a the “mid-sagittal” plane); (ii) the “coronal” or “frontal” plane, being any plane that passes vertically through the body and is perpendicular to the median plane, dividing the body into anterior (front) and posterior (back) sections, and (iii) any “horizontal” or “transverse” or “axial” plane, which pass at right angles to both the median and coronal plane, dividing the body into upper and lower sections.

FIG. 1A shows a human vertebra 10 with pedicles 12 as viewed in a horizontal (transverse) plane. As is commonly known, a pedicle 12 is a basal attachment or bridge portion of the bony vertebra which connects the laminae 14 with the centrum 16. In FIG. 1A, line “M” which bisects the vertebra 10 shows the location of the median or mid-sagittal plane. As further referred to herein, the “pedicle axis” is shown as line “X” which passes from the lamina 14 through the pedicle 12 into the centrum, or vertebral body, 16. It is precisely through this pedicle axis X which a screw, or drill or tap, must be placed during surgery. As can be seen from FIG. 1A, the angle of the pedicle axis X relative to the median plane M is shown as angle “A”, which in the example of FIG. 1A is approximately 25 degrees. Any angle A shall be hereinafter referred to as the “sagittal angle” for a pedicle, which can be understood as the angle of a pedicle relative to the median or mid-sagittal plane M.

FIG. 1B shows the same human vertebra 10 with pedicle 12 as viewed in a sagittal plane. In FIG. 1B, line “T” shows the location of a horizontal or transverse plane. As can be seen from FIG. 1B, the pedicle axis X relative to the transverse plane T is shown as angle “B”, which in the example of FIG. 1B is approximately 17 degrees. Any angle B shall be hereinafter referred to as the “transverse angle” for a pedicle, which can be understood as the angle of a pedicle relative to an axial/horizontal/transverse plane T.

Protractors are commonly known devices for the measurement of angles relative to a reference line, plane or body. A typical protractor level is shown in U.S. Pat. No. 993,912. Protractors for measuring anatomical bodies are also known, such the sternoclavicular joint measuring instrument shown in U.S. Pat. No. 3,047,957, which allows the user to measure one angle of a body relative to a level or gravity vector. Protractors can also utilize plumb elements that utilize the pull of gravity to measure an angle or surface relative to the gravity vector, such as the chest wall angle measuring device shown in U.S. Pat. No. 4,307,517. This patent also shows how a plumb element used in a protractor can be an indicator to show an angular orientation. Another example of this is shown in U.S. Pat. No. 4,358,897, where a single rotational degree of freedom protractor device can be used to measure the angle of a horse's shoulder bone relative to vertical. Surgical devices which have protractors or plumb element-utilizing protractors are shown in U.S. Pat. Nos. 4,733,661 and 5,102,391.

All of the prior art known to applicant however fails to provide a protractor device which accurately and compactly allows a user to orient a surgical element by hand along at least two angles or planes measured from a reference, such as would be necessary to orient or place a surgical element such as a screw into a spinal pedicle. There is a need therefore for a protractor-type device that is simple and easy to use manually, which can permit a surgeon to place a pedicle screw or drill into a pedicle, using at least two accurately oriented angles or planes.

### SUMMARY OF THE INVENTION

The foregoing needs are met, to a great extent, by the present invention, wherein in one aspect an apparatus is provided that in some embodiments allows a surgeon or other user to orient a surgical instrument or device in at least two accurately oriented angles or planes relative to a surgical body to which the instrument is to be applied. The tool can generally be used in any surgical procedure. The utility of the tool of the present invention is demonstrated herein in using said tool to align a surgical instrument relative to the pedicles of the spinal vertebrae.

In accordance with one aspect of the present invention, a tool is provided for aligning an instrument defining a first tool axis. First and second angular scales are disposed on the tool. The tool includes a tool attachment to couple the instrument to the surgical tool. The tool includes a handle defining a second tool axis transverse to the first tool axis, and a hemispherical dome housing. A plumb assembly is rotationally coupled to the hemispherical dome housing to rotate about the second tool axis and to rotate about a third tool axis transverse to the second tool axis.

In accordance with another aspect of the present invention, a tool is provided for aligning an instrument defining a first tool axis. First and second angular scale means are disposed on the tool for indicating first and second angles of the first tool axis relative to first and second reference planes. The tool includes a tool attachment means for coupling the instrument to the surgical tool, a handle means defining a second tool axis transverse to the first tool axis, and a hemispherical dome housing means. A plumb assembly means is rotationally coupled to the hemispherical dome housing to rotate a plumb body and plumb marker about the second tool axis and to rotate about a third tool axis transverse to the second tool axis.

In accordance one embodiment of the present invention, a pedicle protractor tool is provided, having a tool attachment coupling for attaching a surgical instrument aligned along a first tool axis. The tool further includes a dome element having a pivot shaft disposed across a diameter of the dome element aligned along a second tool axis transverse to the first tool axis. The first tool axis and second tool axis together define a first tool plane. A plumb assembly is rotationally fixed on the pivot shaft at a centerpoint of the dome element to permit multi-axis rotation of the plumb assembly about the centerpoint and about both: (i) the second tool axis, and (ii) a third tool axis transverse to the second tool axis. An outer surface of the dome element is marked with a first scale to indicate a degree of rotation of the plumb assembly about the second tool axis. The plumb assembly includes an upper plumb portion proximate an inner surface of the dome element and marked with a second scale to indicate a degree of rotation of the plumb assembly about the third tool axis.

In accordance with yet another aspect of the present invention, a surgical hand tool is provided, having a handle, a bushing defining a channel oriented along a first axis, and a hemispherical dome having a shaft disposed across a diameter at a base of the hemispherical dome, the shaft being oriented along a second axis. A plumb assembly is rotationally coupled to the shaft. The plumb assembly includes a plumb marker rotationally fixed to the shaft, the plumb marker rotatable about the shaft around the second axis. A plumb body is rotationally fixed to the plumb marker, the plumb body being free to rotate about a third axis perpendicular to the second axis.

In accordance with yet another aspect of the present invention, a surgical hand tool for aligning a surgical element relative to at least two reference axes is provided. The tool includes a handle means for gripping the hand tool manually, a bushing means for receiving an elongate surgical element, and a dome means having a base and an exterior surface marked with a first angular scale. A plumb means is fixed to a shaft disposed across the base of the dome means. The plumb means includes an indicator element disposed within the dome means. The plumb means is rotationally coupled to the shaft to permit rotation of the plumb means about two rotational axes. The plumb means includes a surface proximate the dome means marked with a second angular scale. The indicator element is aligned to translate relative to the first and second angular scales by rotation of the plumb means about the two rotational axes.

In accordance with another embodiment of the present invention, a surgical hand tool is provided, including a tool attachment for attaching a surgical tool oriented along a first tool axis, a handle spanning a second tool axis, and a first dome housing defining a cut-out surface area bounded by a perimeter edge of an outer surface of said first dome housing. The perimeter edge defines an indicator edge element. A second dome housing is rotationally coupled to the first dome housing along a first rotation axis transverse to the tool axis. The second dome housing includes first and second angular scales indicated on an outer surface. The first angular scale is disposed to indicate rotation of the second dome housing about the first rotation axis relative to the indicator edge element. The second angular scale is disposed along a cut-out arcuate channel surface area defined by a perimeter edge on the outer surface of said second dome housing. A shaft is disposed across a diameter at a base of the second dome housing. The shaft is oriented along a second rotation axis transverse to the first rotation axis and is oriented in a first tool plane spanned by the first tool axis and second tool axis. A plumb body is rotationally coupled to the shaft to rotate about the second rotation axis. The plumb body includes a plumb marker which translates through the cut-out arcuate channel surface area to indicate rotation of the plumb body about the second rotation axis relative to the second angular scale.

In accordance with yet another aspect of the present invention, a surgical hand tool for aligning a surgical instrument relative to at least two reference axes includes a handle means for gripping the hand tool manually, an attachment means for receiving an elongate surgical instrument, a first dome means having a base and an exterior surface defining an indicator means. The tool includes a second dome means having first and second angular rotation scales disposed on an exterior surface. The second dome means is coupled to the first dome means to rotate about a first rotation axis. The first angular scale indicates rotation of the second dome means about the first rotation axis relative to the indicator means on the first dome means. A plumb means is fixed to a shaft disposed across a base of the second dome means. The plumb means includes an indicator element disposed to indicate rotation of the plumb means relative to the second angular rotation scale on the second dome means.

There has thus been outlined, rather broadly, certain embodiments of the invention in order that the detailed description thereof herein may be better understood, and in order that the present contribution to the art may be better appreciated. There are, of course, additional embodiments of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1A is a view illustrating a vertebra with pedicles as shown in a horizontal or transverse plane.

FIG. 1B is a view illustrating the vertebra with pedicles of FIG. 1A, this time shown in a sagittal plane.

FIG. 2A shows a plan view of a pedicle protractor assembly according to a preferred embodiment of the invention applied to orient a surgical tap, drill or wire to a vertebra, as viewed in a plane parallel to the coronal plane.

FIG. 2B is an enlarged detail view of a circular region of a portion of the pedicle protractor assembly in FIG. 2A, showing the angular scales on the pedicle protractor assembly.

FIG. 3A shows another view of the pedicle protractor assembly of FIG. 2A, as viewed in a plane parallel to a transverse or horizontal plane.

FIG. 3B is an enlarged detail view of a circular region of a portion of the vertebra shown in FIG. 3A, showing a surgical element such as a drill or tap entering a pedicle in said vertebra as oriented and positioned by the pedicle protractor assembly of the present invention.

FIG. 4A shows another view of the pedicle protractor assembly of FIGS. 2A and 3A, this time as viewed in a plane parallel to a sagittal plane.

FIG. 4B is an enlarged detail view of a circular region of a portion of the vertebra shown in FIG. 4A, showing a surgical element such as a drill or tap entering a pedicle in said vertebra as oriented and positioned by the pedicle protractor assembly of the present invention.

FIG. 5 is a perspective view showing the exploded components of a pedicle protractor assembly of the present invention as shown in FIGS. 2A, 3A, and 4A.

FIG. 6 is a perspective view of a plumb assembly forming a part of a pedicle protractor assembly of the present invention.

FIGS. 7A-7C illustrate alternate views of a portion of the plumb assembly upper portion shown in FIGS. 5-6.

FIG. 8A-8B illustrate alternate views of a dome element of a pedicle protractor assembly of the present invention.

FIG. 9 shows a plan view of a pedicle protractor assembly according to another preferred embodiment of the invention, applied to orient a surgical instrument such as a tap, drill or wire to a vertebra, as viewed in a plane parallel to the coronal plane.

FIG. 10 shows another view of the pedicle protractor assembly of FIG. 9, as viewed in a plane parallel to a transverse or horizontal plane.

FIG. 11 shows another view of the pedicle protractor assembly of FIGS. 9 and 10, this time as viewed in a plane parallel to a sagittal plane.

FIG. 12 is a perspective view showing the exploded components of a dome housing and plumb assembly of the pedicle protractor tool assembly shown in FIGS. 9-11.

FIGS. 13A-D show several views of a hemispherical dome housing element included in the pedicle protractor tool assembly shown in FIGS. 9-12.

FIGS. 14A-D show several views of another hemispherical dome housing element included in the pedicle protractor tool assembly shown in FIGS. 9-12.

FIG. 15 shows an angled close-up view of the dome housings and angular scales with indication means for the pedicle protractor tool assembly and constituent elements shown in FIGS. 9-12, 13A-D, and 14A-D, as said tool is being used to orient a surgical instrument into a pedicle of a vertebra.

### DETAILED DESCRIPTION

The invention will now be described with reference to the drawing figures, in which like reference numerals refer to like parts throughout. An embodiment in accordance with the present invention provides a surgical hand tool which allows a surgeon or user to orient a surgical instrument such as a drill, tap, or screw relative to a body in at least two angles or planes, as measured relative to two reference lines or axes or planes.

In accordance with conventional practice, as used herein, the term “proximal” or “proximal end” shall refer to the specified end of a device or its component which is generally closer to the medical personnel handling or manipulating the device as it is intended to be used, and the term “distal” or “distal end” shall refer to the specified end of a device or its component which is opposite the proximal end. Furthermore, as used herein, when used with respect to a spatial element such as a line, axis, or plane, the term “transverse” shall mean perpendicular or at right angles in at least one frame or plane of reference. Also, as used herein, “peripheral” shall mean away from or at an end portion relative to a reference point or line.

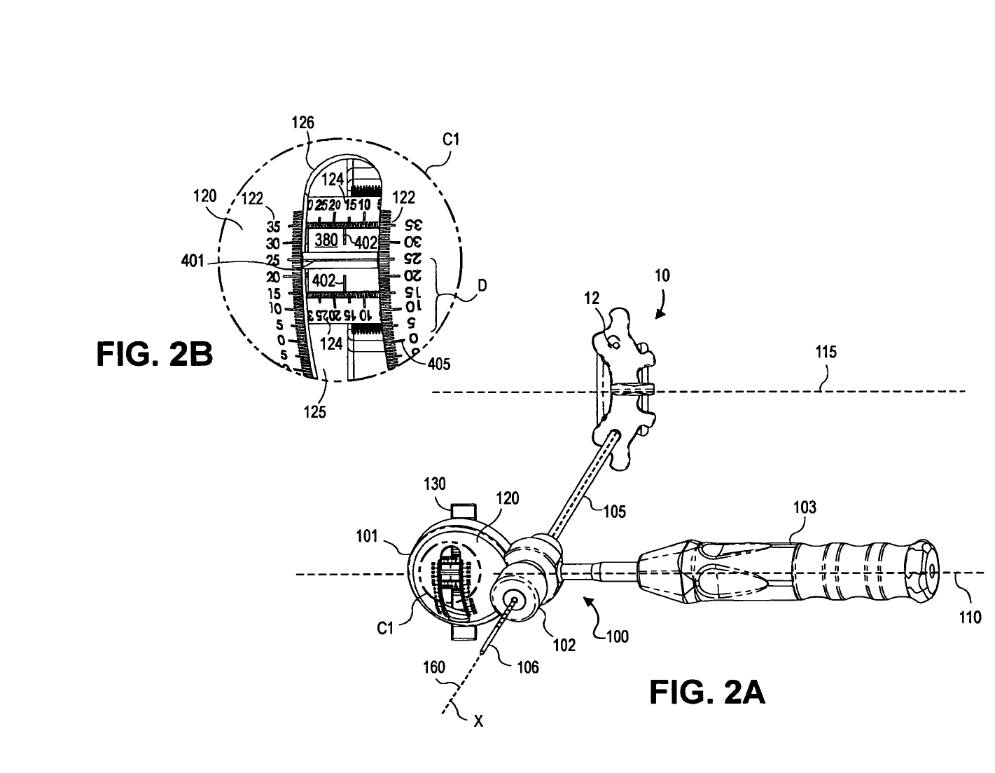

An embodiment of the present inventive apparatus is illustrated in FIG. 2A, showing a pedicle protractor tool or assembly 100, having a dome housing element 101, a “tool attachment coupling” or bushing assembly 102, and a handle element 103 coupled thereto. The pedicle protractor 100 is shown oriented relative to a single vertebra 10, as shown in a planar view which is parallel to the coronal plane of the body. The tool 100 is used to orient a “surgical instrument,” which, as used herein, can be any instrument having a particular longitudinal axis which must be aligned or pointed during a surgical procedure. In FIG. 2A, such surgical instrument is an elongate tubular element 105 such as a drill guide or tap guide, which is disposed through a channel defined by bushing assembly 102 which serves as the tool attachment coupling. A drill shaft 106 is shown protruding through an opening on the upper or top surface of the bushing assembly 102. Said shaft 106 can be axially inserted and slide through instrument tube 105.

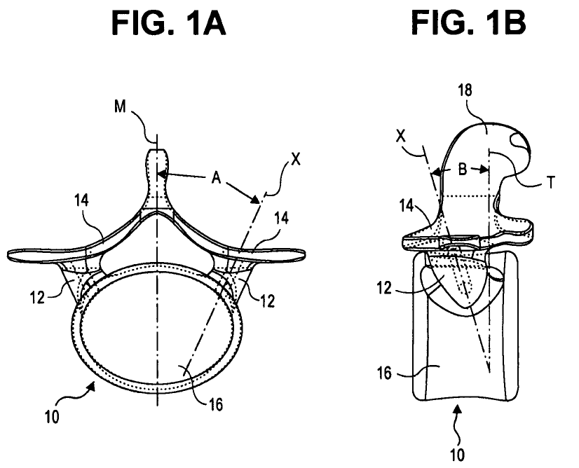

As illustrated in FIG. 2A, the handle 103 is an elongate element which can have a central longitudinal axis 110 aligned through its lengthwise long dimension. A “long axis” 115 is also shown for the body in which the vertebra 10 is disposed, being parallel to an intersection of the coronal plane with the median plane. Long axis 115 can be coincident with line “M” shown in FIG. 1A for a vertebra. The central longitudinal axis 110 can also be referred to as a “handle axis,” which handle axis can be a “tool axis.” The instrument 105 also defines a longitudinal axis 160 which serves as the axis of the instrument 105 which must be aligned by tool 100 during a surgical procedure. Said longitudinal axis 160 of instrument 105 can be referred to herein as a “first tool axis,” while the handle axis 110 of the handle 103 can be referred to herein as a “second tool axis.” In tool 100, when instrument 105 is inserted into the bushing assembly 102, the longitudinal axis 160, or “first tool axis,” is at a right angle or transverse to the handle axis 110, or “second tool axis.” Such first and second tool axes thereby form a “first tool plane” spanned by the overall structural parts of tool 100.

Handle axis 110 can be placed parallel to the long axis 115, to form a beginning reference position for the tool 100 prior to exact positioning of the instrument 105 at the correct angles relative to the vertebral pedicle. The axis of the instrument 105 must be positioned co-linear with the pedicle axis X for any surgical instrument such as a drill or screw to be accurately placed through the pedicle. As can be seen from FIG. 2A, this axis X is formed at a complex three-dimensional angle relative to the body axis 115, and coronal plane. This complex three-dimensional angle can be measured relative to at least two reference planes or lines, by positioning an object or instrument at a first angle relative to a first reference plane or line, and then at a second angle relative to a second reference plane or line.

The tool 100 of the present invention includes a device capable of orienting the surgical instrument 105 relative to at least two reference planes or lines, through two separate angles, which can be measured by two angular scales disposed to be viewed at an upper surface 120 of the dome housing element 101. FIG. 2B is an enlarged detail view of a circular region of a portion of the pedicle protractor tool 100, showing two angular scales 122 and 124 viewable through upper surface 120 of the dome housing element 101 inside reference circle C1. As will be explained in further detail below, upper surface 120 defines a cut-out arcuate channel 125 bounded by a perimeter edge 126. A plumb element or assembly 130 is disposed underneath and within the concave inner volume of the dome housing element 101 and fixed to the tool 100 to rotate about two axes of rotation and thereby indicate an angular position or rotational orientation in at least two angles or lines relative to a reference line or plane, as measured by the angular scales 122 and 124. As further used herein, a “plumb element” or “plumb assembly” or “plumb means” shall include any structure or combination of structures which are coupled to the tool 100 and dome housing element 101 which permit a body to rotate relative to the tool 100 and dome housing element 101 in one or more axes of rotation.

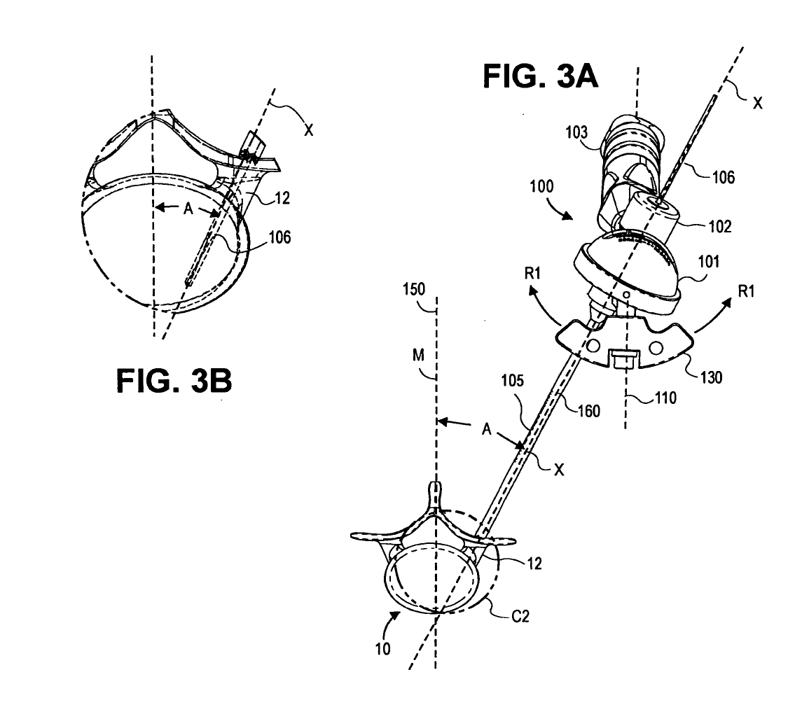

FIG. 3A shows another view of the pedicle protractor assembly 100 of FIG. 2A, as viewed in a plane parallel to a transverse or horizontal plane. Vertebra 10 includes a pedicle 12 having a pedicle axis X centered through the pedicle 12. In FIG. 2A, the pedicle protractor tool 100 includes an elongate surgical instrument 105 in the form an elongate hollow drill guide coupled to the tool attachment coupling assembly in the form of bushing 102. Said instrument 105 has been aligned with pedicle axis X as shown in FIG. 3A such that a drill 106 can be effectively and accurately inserted through the pedicle 12. Median plane M is also shown, through axis 150 co-incident with said plane M. To properly orient surgical instrument 105 relative to the pedicle axis X, a first angle A must be measured relative to a reference plane which is the median plane M (or relative to a reference axis which is axis 150). Angle A is the angle subtended by the central shaft portion of pedicle 12 as more clearly shown in FIG. 3B, an enlarged detail view of a circular region C2 of a portion of the vertebra shown in FIG. 3A, showing a surgical instrument such as a drill or tap 106 entering a pedicle 12.

The present inventive pedicle protractor tool 100 includes a plumb element 130 which allows a user to properly ascertain the angular positioning of tool 100 and its instrument shaft 105 relative to imaginary axes or planes such as the pedicle axis X and median (mid-sagittal) plane M, by measuring the angle A (also referred to herein as the “sagittal angle”) on an indicator or angular scale disposed on the device 100. Plumb element 130 is rotationally fixed to the tool 100 to rotate about at least two axes, in at least two rotational planes. A first rotational degree of freedom is indicated by the arrows R1 shown in FIG. 3A, wherein the plumb element 130 is free to rotate about handle axis 110, in a “first rotational plane” parallel to the plane of the view in FIG. 3A. Said first rotational plane is transverse to the first tool plane spanned by handle axis 110 and the length of instrument 105. As will be explained in further detail below, the rotation of plumb element 130 in this first rotational plane can be measured by the pedicle protractor tool 100 to determine the angle A.

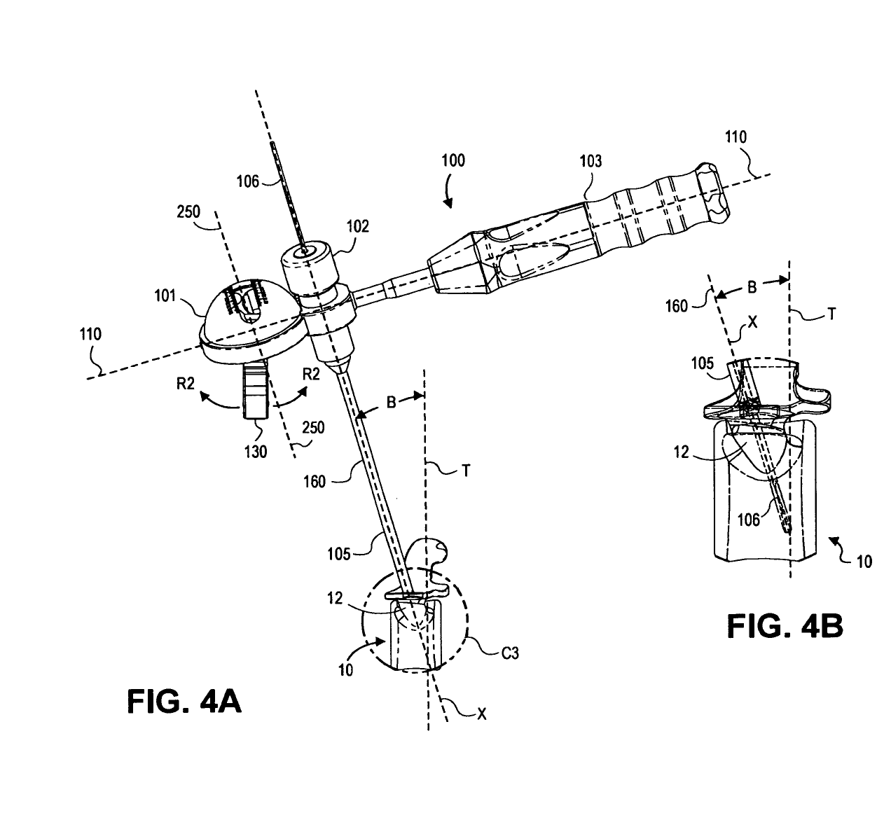

In addition to the angle A, or sagittal angle, relative to the median (mid-sagittal) plane, a pedicle also forms an additional transverse angle relative to a transverse (horizontal) plane. FIG. 4A shows another view of the pedicle protractor assembly 100 of FIGS. 2A and 3A, this time as viewed through a plane parallel to a sagittal plane. FIG. 4B is an enlarged detail view of a circular region C3 of a portion of the vertebra 10 shown in FIG. 4A, showing a surgical instrument such as a drill or tap 106 entering a pedicle 12 in said vertebra 10 as oriented and positioned by the pedicle protractor tool 100. Angle B, which is also referred to herein as the “transverse angle,” is the angle subtended by the pedicle axis X and a transverse plane T, as shown in FIGS. 4A and 4B. Angle B relative to the transverse plane T (or an axis co-incident with it) is used to properly orient the instrument 105 and first tool axis 160 of the tool 100 with the pedicle axis X.

To properly orient the tool 100 and instrument 105 relative to transverse plane T, said angle B must be measured by a user when positioning the tool 100. As discussed above, the pedicle protractor 100 includes a plumb element or assembly 130. In addition to the rotation in the first rotational plane along arrows R1 as shown in FIG. 3A, said plumb assembly 130 can also rotate in a second rotational plane along arrows R2 as shown in FIG. 4A, which second rotational degree of freedom is about a “third tool axis” 250 which is transverse to both the first tool axis 160 and second tool axis 110. Said third tool axis further intersects the second tool axis 110 at a centerpoint of the dome housing 101, where the plumb assembly 130 is fixed to the tool 100.

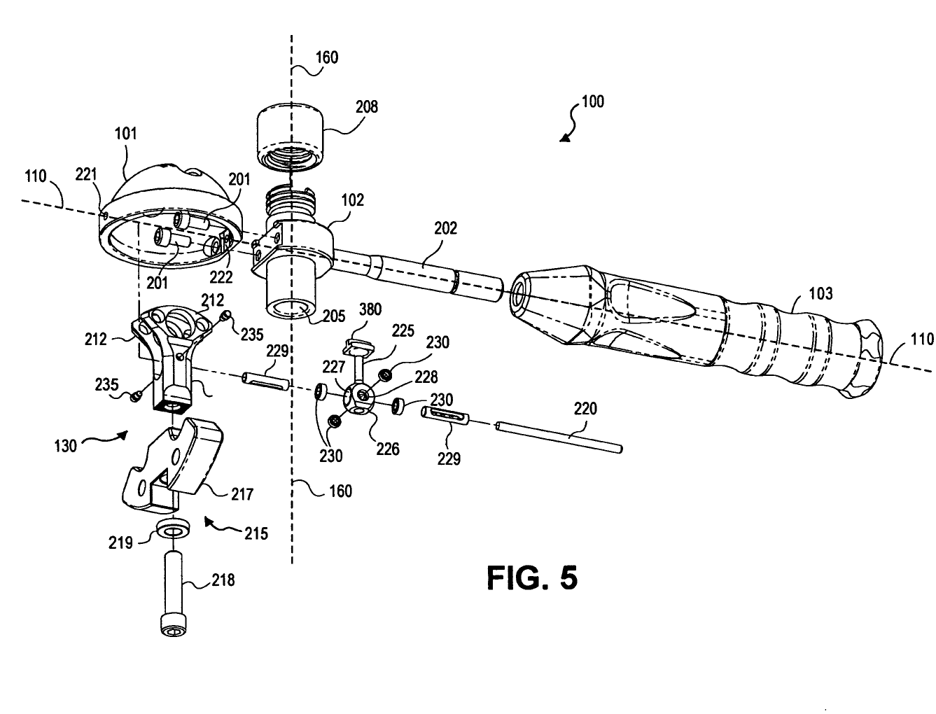

The various structural elements of the surgical hand tool of the present invention are discussed with particular reference to FIG. 5, which is a perspective view showing the exploded components of pedicle protractor tool assembly 100. Tool assembly 100 includes a hollow hemispherical dome element or housing 101, which is fixedly coupled to the bushing assembly 102 by fasteners 201 such as a screw, bolt, or comparable fastening means. Alternatively, dome housing element 101 can be releasably or permanently fixed to bushing 102, in a variety of ways well known to one of skill in the art. Bushing 102 can also be fixed to an elongate shaft 202 which can itself be coupled with, or form an integral part of, the handle 103. In the embodiment shown in FIG. 5, the elongate shaft 202 and handle element 103 are aligned along second tool axis 110 and the dome housing 101 is disposed on the opposite side of bushing 102. The first tool axis 160 is centered through a passage 205 defined or formed by the bushing assembly 102, which is one embodiment of a tool attachment coupling means in the present invention. Bushing assembly 102 further includes a screw cap 208 as shown, to facilitate the fixation and centering of surgical instruments to be disposed through bushing 102 and passage 205 therein, such as a drill, drill guide, tap, etc.

Plumb element or assembly 130 is also shown in more detail in FIG. 5, and includes a plumb body made up of an upper plumb portion 210 which is a forked structure having a pair of wing elements 212, and a lower portion 215. Lower portion 215 includes a plumb weight 217 fixed to upper plumb portion 210 via a fastener 218, such as a screw, bolt, or comparable fastening means. Fastener 218 can be fastened to upper plumb portion 210 with a washer element 219. Alternatively, the plumb body or plumb assembly 130 can be made of a single unitary body instead of an upper plumb portion 210 fastened to lower portion 217, such that each of upper plumb portion 210 and lower portion plumb weight 217 can be integral parts of an overall single plumb assembly. In any embodiment however, the mass of the lower portion plumb weight 217 is considerably higher than the mass of upper plumb portion 210, such that the plumb assembly 130 will properly function in the presence of gravity.

Plumb assembly 130 is fixed to the tool 100 via a pivot pin or pivot shaft 220 disposed across a diameter of the dome housing element 101 between points 221 and 222. In the embodiment shown in FIG. 5, pivot shaft 220 is aligned with second tool axis 110, in line with the handle 103 and elongate shaft 202. A plumb marker element 225 having a central pivot portion 226 defines at least two orthogonal channels 227 and 228. Pivot shaft 220 is disposed through channel 227 in a direction parallel to second tool axis 110. A pair of sleeve elements 229 can be disposed around the pivot shaft 220 on either side of central pivot portion 226 of the plumb marker element 225. Though not essential, bearing elements 230 can be inserted into each orifice of channels 227 and 228 and around pivot shaft 220 to facilitate (with greatly reduced friction) rotation of the plumb marker element 225 about the pivot shaft 220 and hence about the second tool axis 110. Plumb pin elements 235 couple the plumb upper portion 210, and hence the entire plumb assembly 130 including the plumb weight 217, to both the plumb marker element 225 and the pivot shaft 220. However, because the plumb pins 235 are inserted into channel 228 which is orthogonal to channel 227, the plumb pins are therefore oriented to allow for rotation of the plumb assembly 130 about an other axis transverse to the second tool axis 110, which other axis is the third tool axis 250 shown in FIG. 4A. The plumb assembly 130 therefore is rotationally fixed on the pivot shaft 220 to permit multi-axis rotation of the plumb assembly 130 about both the second tool axis 110 and third tool axis 250 which is co-incident through the two plumb pins 235 and through the channel 228 on central pivot portion 226.

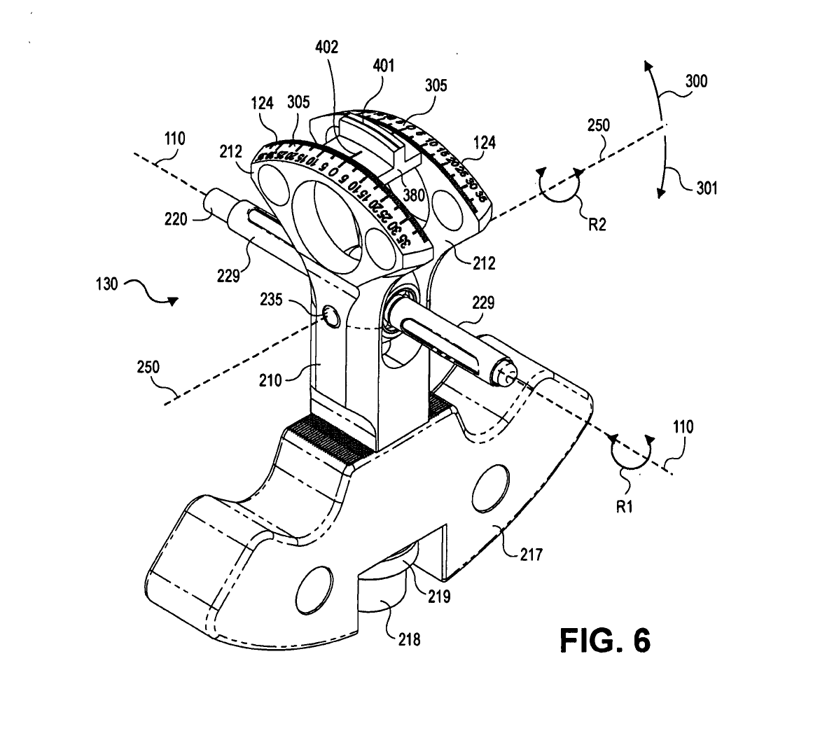

FIG. 6 is a perspective view of the plumb assembly 130, shown unattached to dome element 101 of tool 100. The “third tool axis” 250 is shown running through the plumb pins 235 and transverse to second tool axis 110. Thus, the plumb assembly 130 can rotate about second tool axis 110 in the direction shown by arrows R1 in FIG. 6, and simultaneously rotate about third tool axis 250 in the direction of arrows R2 shown in FIG. 6. While the second tool axis 110 is always fixed relative to the rest of the tool assembly 100 by virtue of the pivot shaft 220 being fixed to dome element 101, as the plumb assembly 130 rotates about second tool axis 110, the third tool axis 250 will rotate about second tool axis 110, and hence change direction relative to a fixed reference frame around the tool assembly 100. Thus, the direction of third tool axis 250 changes with the rotation of plumb assembly 130 about second tool axis 110, in the direction of arrows 300 and 301 as shown in FIG. 6. However, third tool axis 250 will always remain transverse to second tool axis 110.

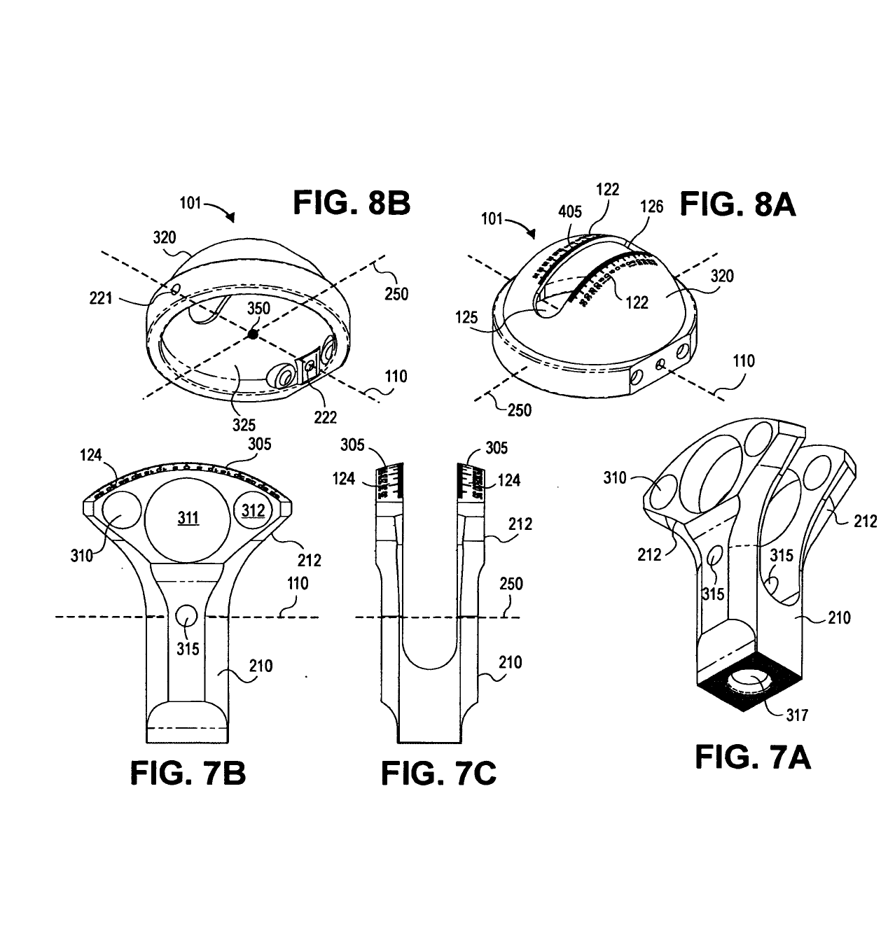

The angular scale 124 is printed on an upper, peripheral surface 305 of wing elements 212 of forked upper plumb portion 210. FIGS. 7A-7C illustrate alternate views of a portion of the plumb assembly forked upper plumb portion 210 shown in FIGS. 5-6. Mass lightening holes 310, 311, 312 are bored through the structure of upper plumb portion 210 on both wings 212 to provide additional balancing and allow for the plumb upper portion 210 to be significantly lower in mass and hence lighter than the plumb weight 217. Holes 315 in FIGS. 7A-7B show where the plumb pins 235 are placed to rotationally fix the upper plumb portion 210 to the pivot portion 226 of plumb marker 225. Second tool axis 110 is shown in FIG. 7B and third tool axis 250 is shown in FIG. 7C in the respective positions each axis would be when upper plumb portion 210 is fixed to plumb assembly 130 as shown in FIGS. 5-6. An opening 317 is formed on the lower underside of upper plumb portion 210 in FIG. 7A to indicate where a fastening means may be inserted to coupled plumb weight 217.

FIG. 8A-8B illustrate alternate views of dome housing element 101 of the pedicle protractor tool 100. Dome housing 101 is a hemispherical dome or circular half-shell that is hollow, having an outer surface 320 on the top upper portion, and an inner surface 325 on the other side of the dome shell. Openings or holes 221 and 222 show where the pivot shaft 220 is inserted within the dome. When the plumb assembly 130 is fully assembled and coupled to the pivot shaft 220, the curved upper peripheral surfaces 305 of the planar wing elements 212 will be proximate to and just below the inner surface 325 of the dome element 101. Indeed, the curved edges of upper surfaces 305 define a spherical arc mated to inner surface 325 of the hemispherical dome 101.

Second tool axis 110 and third tool axis 250 are shown in FIGS. 8A-8B in the respective positions each axis would be at when the tool assembly 100 is fully assembled with the dome element 101. As can be seen in FIG. 8B, Second tool axis 110 and third tool axis 250 intersect at a “centerpoint” 350, which point is where the plumb assembly 130 is rotationally fixed to the pivot shaft 220. The dome housing 101 and pivot shaft are therefore sized such that the center of pivot shaft 220 will coincide with centerpoint 350. As can be further seen in FIG. 8B, due to the circular symmetry of dome 101, the pivot shaft 220 when inserted between points 221 and 222 lies along a “diameter” of the hemispherical dome housing 101. The curved upper peripheral surfaces 305 of the planar wing elements 212 are peripheral to the centerpoint 350.

In FIG. 8B, the upper, “outer” surface of dome element 101 includes a channel 125 that runs through an arc. Channel 125 includes a perimeter edge 126 around which angular scale 122 is printed. Turning back to FIGS. 5 and 6, plumb marker element 225 is fixed on the pivot shaft 220 at the centerpoint thereof and at the centerpoint 350 of the dome housing 101 and includes a peripheral portion or peripheral wing 380 which has two indicator lines 401 and 402 marked thereon. As the plumb body assembly 130 rotates about the centerpoint 350 and about the pivot shaft 220, the peripheral wing 380 will move relative to the angular scales 122 and 124. The indicator line 401 can be used to measure the degree of rotation of plumb body 130 about second tool axis 110 using angular scale 122. And indicator line 402 can be used to measure the degree of rotation of upper plumb portion 210 about the third tool axis 250 using angular scale 124. FIG. 2B shows an enlarged view of the two indicator lines 401 and 402 when the plumb body 130 has been rotated about both the second and third tool axes 110 and 250. The degree of rotation of the plumb body about second tool axis 110 is shown by bracket “D”, where indicator line 401 on the peripheral surface 380 of plumb marker 225 shows a rotation of 25 degrees from the zero point of angular scale 122. Indicator line 402 shows a rotation of about 17 degrees about the third tool axis 250 from the zero point of angular scale 124.

The rotations of plumb assembly 130 indicated on angular scales 122 and 124 are equal to angles A and B, respectively, as shown in FIGS. 3A-3B and 4A-4B. In FIG. 8A, it can be seen that the zero point 405 of angular scale 122 is positioned at the direct apex of the upper outer surface of dome 101. When the bottom circular span of hemispherical dome housing 101 is directly level relative to gravity (i.e. perpendicular to the gravity vector), the zero point 405 of angular scale 122 is directly in line with gravity. The freely rotatable plumb assembly 130 on tool 100 can be calibrated to point to both the zero points on the two angular scales 122 and 124 when the median and transverse plane T of the body to which the surgical instrument 105 is to be applied is aligned with a gravity vector “g”, such that body long axis 115 is perpendicular to gravity, i.e., a patient is lying down. As shown in FIG. 3A, as the tool 100 is tilted to form an angle A between the instrument shaft 105 and first tool axis 160 relative to the median/mid-sagittal plane M, where the axis 150 of said plane M is aligned with gravity, the sagittal angle A is equal to the degree of rotation of plumb body assembly 130 about the second tool axis 110 as measured from the zero point 405 on the “first” angular scale 122. Similarly, as shown in FIG. 4A, as the tool 100 is tilted to form an angle B between instrument shaft 105 and first tool axis 160 relative to transverse plane T, where said plane T is also aligned with gravity, the transverse angle B is equal to the degree of rotation of plumb body 130 about the third tool axis 250.

Thus, the surgical tool of the present invention allows a user to align a surgical instrument in at least two angles from a reference line or plane. In the case of the rotation of plumb body 130 about the third tool axis 250, the “first” reference plane is a plane transverse to second tool axis 110 and can be the transverse plane T. In the case of the rotation of plumb body 130 about the second tool axis 110, the “second” reference plane is a plane parallel to the second tool axis 110 and can be the median plane M. As will be understood by those of skill in the art, the first and second reference planes are transverse to each other.

Although an example of an application of surgical tool 100 is shown to measure the angles relevant to insertion of elements into the pedicles of a vertebra, such an application is but one embodiment or aspect of the present invention. It will be appreciated that other applications or embodiments are possible. Indeed, the present invention can be used whenever two angles must be measured by a hand tool, the angles being measured relative to reference planes that are transverse to each other. Furthermore, although the surgical tool of the present invention is useful to conduct surgery, it can also be used in a variety of other industries or activities, wherever an element must be aligned in two angles.

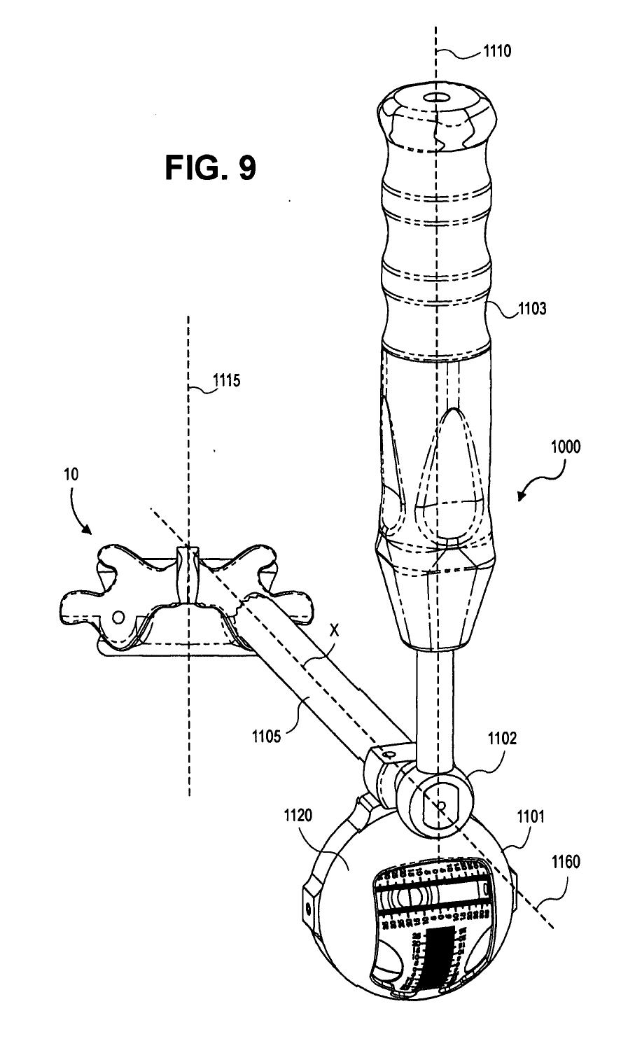

Another embodiment of the present inventive apparatus is illustrated in FIG. 9, showing a pedicle protractor tool or assembly 1000, having a dome housing 1101, a tool attachment such as a bushing assembly 1102, and a handle element 1103 coupled thereto. The pedicle protractor 1000 is shown oriented relative to a single vertebra 10, as shown in a planar view which is parallel to the coronal plane of the body. A surgical instrument or element 1 105, shown in FIG. 9 as an elongate tubular element such as a drill guide or tap guide, can be disposed through a channel or passage disposed in the tool attachment 1102, which instrument 1105 can be oriented by the user holding the tool 1000 with handle 1103.

As illustrated in FIG. 9, the handle 1103 is an elongate element which can have a central longitudinal axis 1110 which is aligned through the lengthwise long dimension of the overall tool 1000. A “long axis” 1115 is also shown for the body in which the vertebra 10 is disposed, being parallel to an intersection of the coronal plane with the median plane. Long axis 1115 can be coincident with line “M” shown in FIG. 1A for a general vertebra. The central longitudinal axis 1110 can also be referred to as a “handle axis” or “tool axis.” The tool 1000 further defines a longitudinal axis 1160 aligned with the surgical instrument 1105. When instrument 1105 is inserted into the tool attachment or bushing assembly 1102, the longitudinal axis 1160 of the elongate tube 1 105 is at a right angle or transverse to the handle or tool axis 1110. Element 1 105 and its longitudinal axis 1160, together with handle axis 1110, thereby form a “first tool plane” generally spanned by the overall structural parts of tool 1000.

Handle axis 1110 can be placed parallel to the long axis 11 15 of the body, to form a beginning reference position for the tool 1000 prior to exact positioning of the instrument 1105 at the correct angles relative to the vertebral pedicle. The longitudinal axis 1160 of the instrument 1105 must be positioned co-incident with the pedicle axis X for any surgical instrument such as a drill or screw to be accurately placed through the pedicle, as shown in FIG. 9. As with the previously discussed embodiments, the tool 1000 of the present invention includes a mechanism for orienting the instrument 1105 relative to at least two reference planes or lines, through two separate angles, which can be measured by two angular scales disposed to be viewed through a portion of an upper surface 1120 of the dome housing 1101. The two angular scales in tool 1000 are oriented differently than those in tool 100 discussed above, although each tool measures the same set of angles formed by the pedicle axis X. Part of the difference lies in the structure of the rotating plumb assembly on tool 1000 shown in FIG. 9.

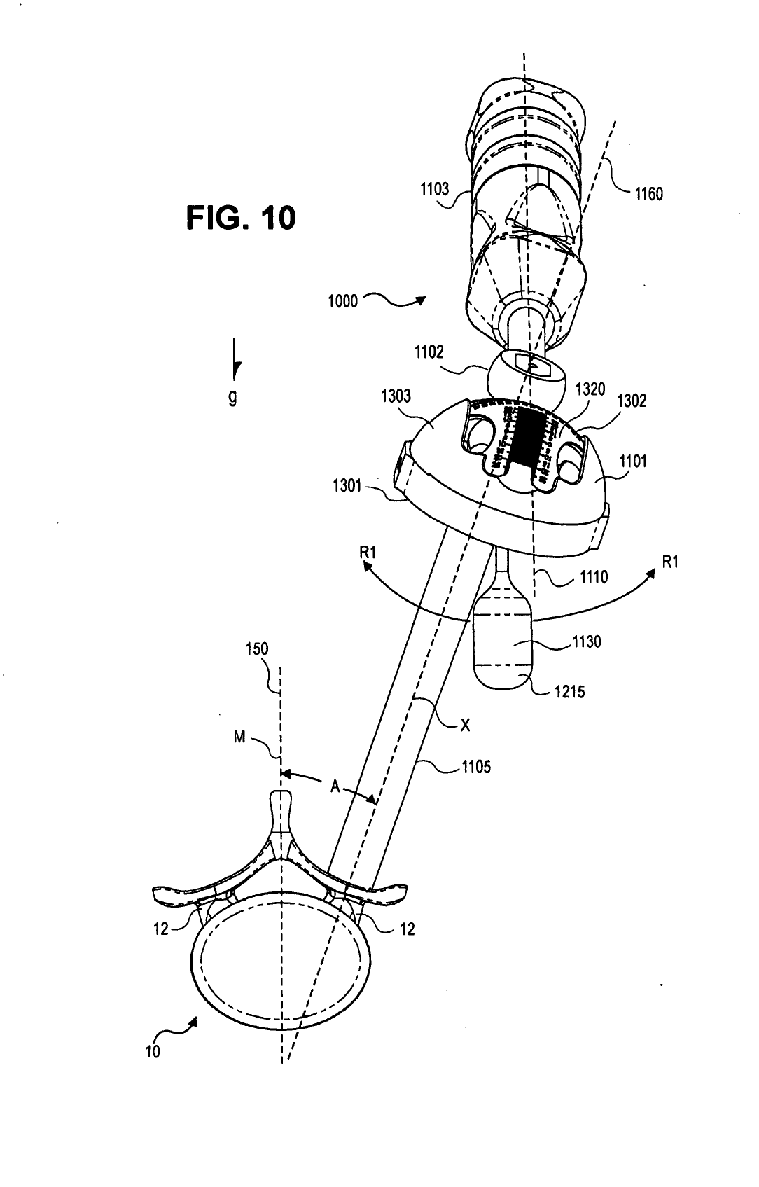

FIG. 10 shows another view of the pedicle protractor assembly 1000 of FIG. 9, as viewed in a plane parallel to a transverse or horizontal plane of the body. Vertebra 10 includes a pedicle 12 having a pedicle axis X centered through a pedicle 12. In FIG. 10, the pedicle protractor tool 1000 has an elongate surgical instrument 1105 inserted through the bushing or tool attachment hub 1102. As further used herein, a “tool attachment” can be any kind of a hub or bushing on tool 1000 as shown in FIGS. 9 and 10, or which simply couples one object with another to form a single tool assembly. Said instrument 1105 has been aligned with pedicle axis X. Median plane M is also shown, through axis 150 co-incident with said plane M. To properly orient instrument 1105 relative to the pedicle axis X, a first angle A must be measured relative to a reference plane (which is the median plane M) or relative to a reference axis (which is axis 150).

The present inventive pedicle protractor tool 1000 includes a plumb element or assembly 1130 which allows a user to properly ascertain the angular positioning of tool 1000 and its instrument shaft 1105 relative to imaginary axes or planes such as the pedicle axis X and median (mid-sagittal) plane M, by measuring the angle A, or sagittal angle, on an indicator or angular scale disposed on the device 1000. As further used herein, a “plumb element” or “plumb assembly” or “plumb means” shall include any structure or combination of structures which are coupled to the tool 1000 and dome housing element 1101 which permit a body to rotate relative to the tool 1000 and dome housing element 1101 in one or more axes of rotation. Plumb element 1130 of the plumb assembly is rotationally fixed to the tool 1000 to rotate about at least two axes, in at least two rotational planes. A first rotational degree of freedom is indicated by the arrows R1 shown in FIG. 10, wherein the plumb element 1130 is free to rotate about an axis co-planar to handle axis 1110, in a “first rotational plane” parallel to the plane of the view in FIG. 10. The length of instrument 1105 thereby forms a “first tool axis” labeled as 1160, which is coincident with the pedicle axis X when the tool 1000 has been properly aligned with the pedicle axis. The handle axis 1110 can also therefore be referred to as a “second tool axis.” The first tool axis 1160 and second tool axis 1110 are transverse (at right angles) to each other. As will be described in more detail below, plumb element 1130 is fixed to tool 1000 to rotate about a rotational axis co-planar to the second tool axis 1110 in the first rotational plane spanned by the direction of arrows R1 in FIG. 10. As will be explained in further detail below, the rotation of plumb element 1130 in this first rotational plane can be measured by the pedicle protractor tool 1000 to determine the angle A.

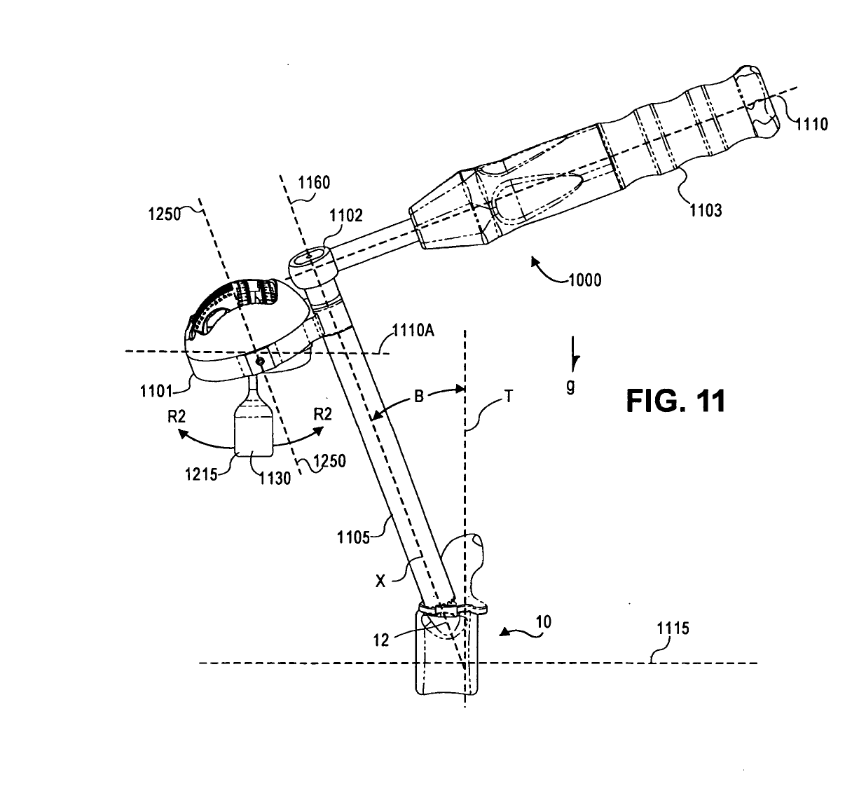

FIG. 11 shows another view of the pedicle protractor assembly 1000 of FIGS. 9 and 10, this time as viewed in a plane parallel to a sagittal plane. Angle B, which is referred to herein as the transverse angle, is the angle subtended by the pedicle axis X and a transverse plane T. Angle B is formed relative to the transverse plane T (or an axis co-incident with it) when the instrument shaft 1105, along the first tool axis 1160, is aligned with pedicle axis X. To properly orient the tool 1000 and instrument 1105 relative to transverse plane T, said angle B must be measured by a user when positioning the tool 1000. As discussed above, the pedicle protractor 1000 includes a plumb element or assembly 1130. In addition to the rotation in the first rotational plane along arrows R1 as shown in FIG. 10, said plumb assembly 1130 can also rotate in a second rotational plane along arrows R2 as shown in FIG. 11, which second rotational degree of freedom is about a “third tool axis” 1250 which is transverse to both the first tool axis 1160 and second tool axis 1110. Said third tool axis further passes through a centerpoint of the dome element 1101, and passes through where the plumb element 1130 is fixed to the tool 1000, as explained more fully below.

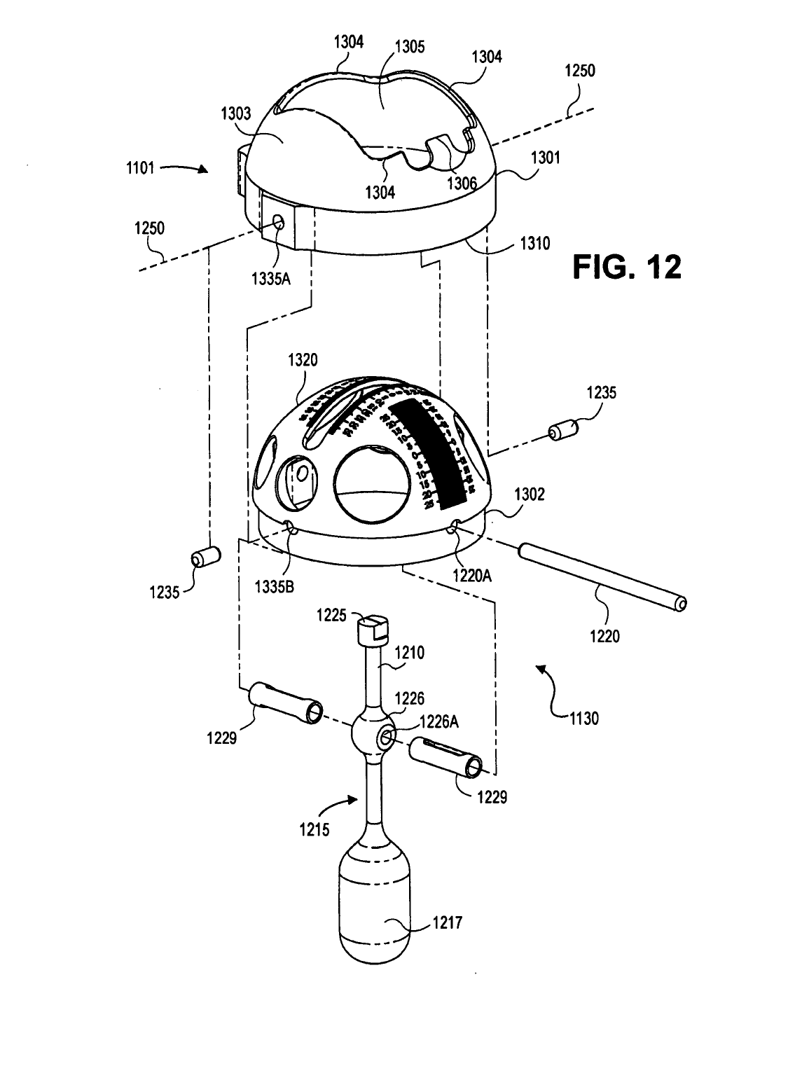

The various structural elements of the surgical hand tool 1000 of the present invention are discussed with particular reference to FIG. 12, which is a perspective view showing the exploded components of the dome housing and plumb assembly of the pedicle protractor tool assembly 1000 shown in FIGS. 9-11. Dome housing 1101 includes a first hollow hemispherical dome housing element 1301. The plumb assembly includes a second hollow hemispherical dome housing element 1302, as shown in FIG. 12. As used herein, a “dome housing” shall mean any structure whose outline generally fits a hemispherical dome shape, but which can have any surface, including a surface which defines one or more cut-out areas which are empty. First hollow hemispherical dome housing element 1301 includes an outer surface 1303 which defines a perimeter edge 1304 which defines a cut-out area 1305. Said perimeter edge 1304 is shaped to have at least one lip-shaped portion 1306 which serves as an indicator edge or element. The first hemispherical dome housing element 1301 is hollow in that the element is a shell structure, into which another hemispherical shell in the form of hemispherical dome housing element 1302 fits inside.

As shown in FIG. 12, second hollow hemispherical dome housing element 1302 is sized to fit closely inside the concave inner surface 1310 of first hemispherical dome housing element 1301, such that the outer surface 1320 of second hemispherical dome housing element 1302 abuts in close proximity with said inner surface 1310 when the tool 1000 is fully assembled, as is best illustrated in FIG. 10. Second hemispherical dome housing element 1302 is coupled to first second hemispherical dome housing element 1301 via two pins 1235 which fit though: (i) a first pair of openings 1335a defined on diametrically opposite points across a diameter on first hemispherical dome housing element 1301, and (ii) a second pair of openings 1335b defined on diametrically opposed points across a diameter on second hemispherical dome housing element 1302. As can be seen in FIG. 12, the third tool axis 1250 runs through the openings 1335a on first hemispherical dome housing element 1301, as well as through openings 1335b on second hemispherical dome housing element 1301 when the tool 1000 is fully assembled.

Plumb element or assembly 1130 is also shown in more detail in FIG. 12, and includes a plumb body 1215 made up of an upper plumb portion 1210 and a lower plumb portion or plumb weight 1217. The entire plumb body 1215 can be a continuous single piece or unitary body, although alternative embodiments can include subassemblies for the plumb body 1215 made up of two or more pieces fastened or fixed together. In any embodiment however, the mass of lower portion plumb weight 1217 is considerably higher than the mass of upper portion 1210, such that the plumb body 1215 and overall plumb assembly 1130 will properly function in the presence of gravity. Upper plumb portion 1210 further includes a plumb marker element 1225 at its tip, having an indicator line disposed thereon.

Plumb body 1215 is fixed to the tool 1000 and dome housing 1101 and first hemispherical dome housing element 1301 via a pivot pin or pivot shaft 1220 disposed across a diameter of the second hemispherical dome housing element 1302 through a pair of diametrically opposed openings 1220a which are defined by second hemispherical dome housing element 1302 across a diameter at the base of said housing element 1302. When the tool 1000 is fully assembled, pivot shaft 1220 can be initially parallel with second tool axis 1110, in line with the handle 1103, prior to any rotation of dome housing element 1302 relative to dome housing element 1301 about axis 1250. A pair of sleeve elements 1229 can be disposed around the pivot shaft 1220 on either side of central pivot portion 1226 defined on the plumb body 1215 between the plumb upper portion 1210 and plumb lower portion 1217. Central pivot portion 1226 defines a channel 1226a though which the pivot pin 1220 is disposed. Bearing elements can be inserted into the channel 1226a through which pivot shaft 1220 is inserted to facilitate (with greatly reduced friction) rotation of the plumb body 1215 about the pivot shaft 1220. The plumb body 1215 is rotationally fixed on the pivot shaft 1220 to permit multi-axis rotation of the overall plumb assembly 1130 about both an axis parallel to the second tool axis 1110 and the third tool axis 1250, due to the particular rotational coupling of second hemispherical dome element 1302 with first hemispherical dome element 1301.

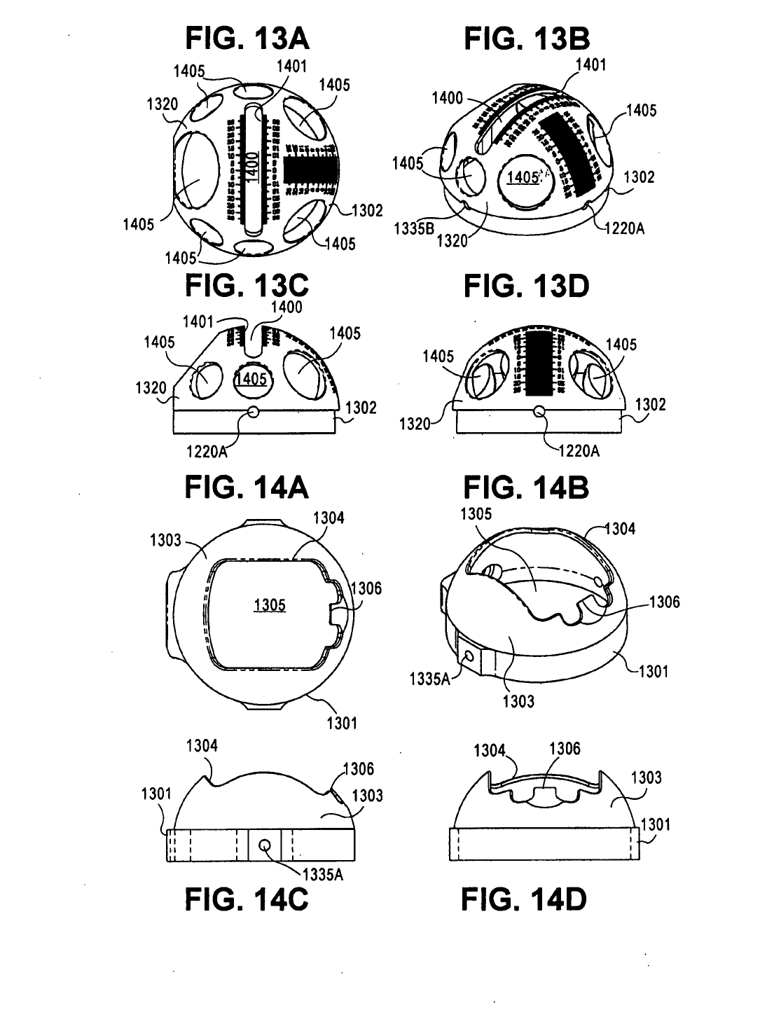

FIGS. 13A-D show several views of the second hemispherical dome housing element 1302, showing the outer surface 1320 which defines a cut-out arcuate channel surface area 1400 defined by a perimeter edge 1401. A plurality of additional cut-out portions of outer surface 1320 are defined by said surface, which additional cut-out portions can be of any shape, but are shown as circular holes 1405. These circular holes 1405 reduce the overall weight of the shell structure 1302 and help balance it when rotating about its rotation axis. Arcuate channel 1400 extends as best shown in FIGS. 13A and 13C along a centerline or diametric plane of symmetry dividing the overall circular outline shape of the overall hemispherical shell housing 1302.

FIGS. 14A-D show several views of the first hemispherical dome housing element 1301, showing the outer surface 1303 which defines the perimeter edge 1304 which defines the cut-out area 1305 and the at least one lip-shaped portion 1306 which serves as an indicator edge or element.

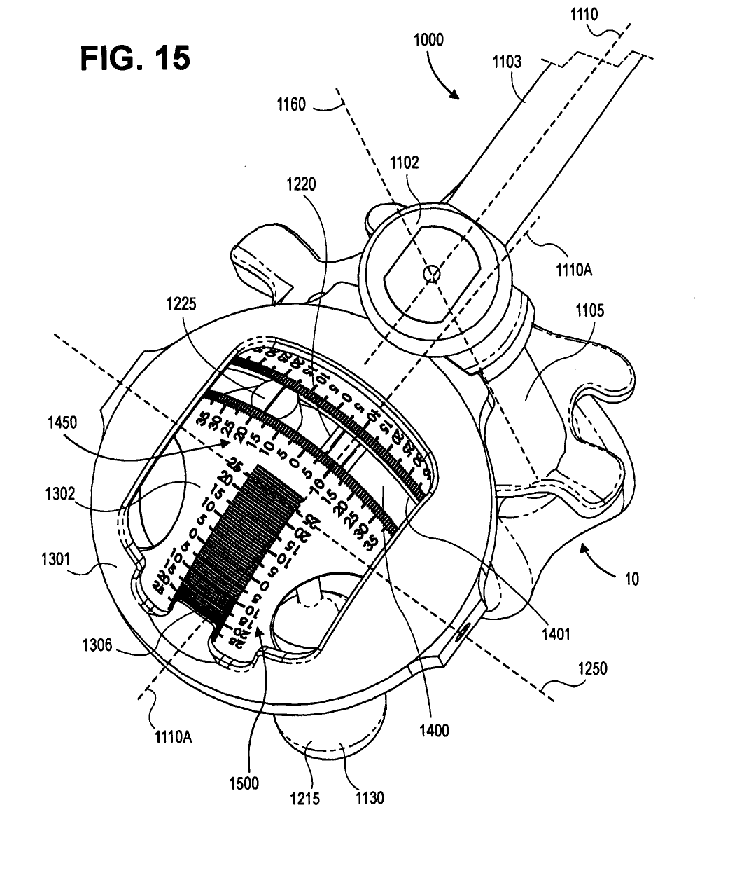

FIG. 15 shows an angled close-up view of the dome housings and angular scales with indication means for the pedicle protractor tool assembly and constituent elements shown in FIGS. 9-12, 13A-D, and 14A-D, as said tool is being used to orient a surgical instrument into a pedicle of a vertebra. Two angular scales 1450 and 1500 are disposed on the outer surface of second hemispherical dome housing element 1302. Each scale includes a pair of angular rotation gradation markings, shown in degrees in FIG. 15, with major scale markings including a numeral indicator every five degrees. A zero point is indicated on each scale 1450 and 1500, with each scale extending for a range beyond either side or rotational direction of said zero point.

Angular rotational scale 1500 indicates the relative rotation of second dome housing 1302 and plumb assembly 1130 about third tool axis 1250, which axis runs through the pins that rotationally couple the second dome housing 1302 to the first dome housing 1301. When handle 1103 is used to tilt tool 1000 as shown in FIG. 11 to create an angle B relative to the transverse plane T running though a vertebra (which transverse plane T is shown aligned with a gravity vector “g”, such that body long axis 1115 is perpendicular to gravity, i.e., a patient is lying down), the inner, second dome housing 1302 and plumb assembly 1130 will rotate relative to the outer, first dome housing 1301, and the resulting rotation will be equal to angle B. Such degree of rotation of angle B is shown on the angular scale 1500 by measuring the scale with indicator lip element 1306. The zero point of angular scale 1500 is thus calibrated for when angle B is zero, and the handle 1103 with second tool axis 1110 is parallel to the body long axis and transverse to the transverse plane T. Since the plumb body 1215 of the plumb assembly 1130 is fixed to the second dome housing 1302, it will also rotate with said housing 1302 and plumb assembly 1130 relative to the first dome housing 1301 (and the rest of the tool 1000), to remain pointing downwards in the direction of gravity g as shown in FIG. 11.

Angular rotational scale 1450 indicates the relative rotation of the plumb body 1215 about axis 1110a which runs though the pivot pin or shaft 1220 shown in FIG. 12 (which shaft 1220 is also indicated in FIG. 15 as viewable through the arcuate cut-out channel 1400 on second dome housing 1302). When the tool 1000 is rotated to form angle A as shown in FIG. 10, said plumb body 1215 rotates about the axis 1110a which is coincident with the shaft 1220 around which said plumb body is rotationally coupled. As tool 1000 is tilted to form angle B relative to gravity, axis 1110a will change direction relative to second tool axis 1110, but both axes 1110a and 1110 remain coplanar with first tool axis 1160 at all times, and thereby all of axes 1110, 1110a, and 1160 remain in the first tool plane. An indicator line on plumb marker element 1225 which translates through the arcuate channel 1400 and provides the reference to measure the degree of rotation of plumb body 1215 relative to angular scale 1450, which rotation is equal to the angle A formed by the longitudinal axis 1160 of instrument 1105 attached to the tool 1000 relative to the median plane M, when said instrument 1105 is aligned with the pedicle axis X.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A tool for aligning an instrument defining a first tool axis, comprising:
first and second angular scales disposed on the tool;
a tool attachment coupling the instrument to the surgical tool;
a handle defining a second tool axis transverse to the first tool axis;
a hemispherical dome housing;
a plumb assembly rotationally coupled to the hemispherical dome housing to rotate about the second tool axis and to rotate about a third tool axis transverse to the second tool axis.

2. The surgical tool of claim 1, wherein the plumb assembly includes a plumb marker to indicate a degree of rotation of the plumb assembly about the second tool axis relative to the first angular scale, and the second angular scale indicates a degree of rotation of the plumb assembly about the third tool axis.

3. A tool for aligning an instrument defining a first tool axis, comprising:
first and second angular scale means disposed on the tool for indicating first and second angles of the first tool axis relative to first and second reference planes;
a tool attachment means for coupling the instrument to the surgical tool;
a handle means defining a second tool axis transverse to the first tool axis;
a hemispherical dome housing means;
a plumb assembly means rotationally coupled to the hemispherical dome housing to rotate a plumb body and plumb marker about the second tool axis and to rotate about a third tool axis transverse to the second tool axis.

4. A pedicle protractor tool, comprising:
a tool attachment coupling for attaching a surgical instrument aligned along a first tool axis;
a dome element having a pivot shaft disposed across a diameter of the dome element aligned along a second tool axis transverse to the first tool axis, the first tool axis and second tool axis together defining a first tool plane; and
a plumb assembly rotationally fixed on the pivot shaft at a centerpoint of the dome element to permit multi-axis rotation of the plumb assembly about the centerpoint and about both:
(i) the second tool axis, and
(ii) a third tool axis transverse to the second tool axis;
an outer surface of the dome element being marked with a first scale to indicate a degree of rotation of the plumb assembly about the second tool axis;
the plumb assembly having an upper plumb portion proximate an inner surface of the dome element and marked with a second scale to indicate a degree of rotation of the plumb assembly about the third tool axis.

5. The pedicle protractor tool of claim 4, further comprising:
an elongate handle element, wherein the elongate handle element is substantially aligned along the second tool axis parallel to the pivot shaft.

6. The pedicle protractor tool of claim 4,
wherein the upper plumb portion comprises a forked structure having a pair of wing elements each having a peripheral surface proximate the inner surface of the dome element, the second scale being marked on said peripheral surfaces.

7. The pedicle protractor tool of claim 6, further comprising:
a plumb marker element disposed between the pair of wing elements and rotationally fixed on the pivot shaft at the centerpoint of the dome element to rotate about the second tool axis, the plumb marker having a peripheral portion with a marking to indicate the degree of rotation of the plumb element about the second tool axis relative to the first scale.

8. The pedicle protractor tool of claim 7,
wherein the dome element defines a channel extending through an arc centered along a first rotational plane through which the plumb marker element rotates about the second tool axis, the peripheral portion of the plumb marker element being disposed to rotationally translate through the channel.

9. The pedicle protractor tool of claim 8,
wherein the first scale is marked on a portion of a perimeter on the outer surface of the dome element defining the channel.

10. The pedicle protractor tool of claim 4, further comprising:
a plumb weight disposed as a lower portion of the plumb assembly opposite to the upper plumb portion, the plumb weight having a mass that is substantially larger than a mass of the upper portion of the plumb element.

11. A surgical hand tool, comprising:
a handle;
a bushing defining a channel oriented along a first axis;
a hemispherical dome having a shaft disposed across a diameter at a base of the hemispherical dome, the shaft being oriented along a second axis;
a plumb assembly rotationally coupled to the shaft, the plumb assembly having:
a plumb marker rotationally fixed to the shaft, the plumb marker rotatable about the shaft around the second axis,
a plumb body rotationally fixed to the plumb marker, the plumb body being free to rotate about a third axis perpendicular to the second axis.

12. The surgical hand tool of claim 11,
wherein the plumb body includes an upper section and a plumb weight, the plumb body upper section including at least one planar wing element extending away from a centerpoint of the shaft into a hollow inner volume defined by the hemispherical dome, the at least one planar wing element having a curved edge peripheral to the centerpoint defining an arc mated to an inner surface of the hemispherical dome.

13. The surgical hand tool of claim 12,
wherein the curved edge includes markings to indicate angular rotation of the plumb body about the third axis relative to a reference plane.

14. The surgical hand tool of claim 11, wherein the plumb marker includes a peripheral surface extending away from a centerpoint of the shaft through an arcuate channel defined by the hemispherical dome, the arcuate channel having a angular scale disposed around a perimeter thereof, the peripheral surface of the plumb marker having a reference line to indicate relative to the angular scale a rotation of the plumb body about the shaft and second axis.

15. A surgical hand tool for aligning a surgical element relative to at least two reference axes, comprising:
a handle means for gripping the hand tool manually;
a bushing means for receiving an elongate surgical element;
a dome means having a base and an exterior surface marked with a first angular scale;
a plumb means fixed to a shaft disposed across the base of the dome means, the plumb means having an indicator element disposed within the dome means, the plumb means being rotationally coupled to the shaft to permit rotation of the plumb means about two rotational axes, the plumb means having a surface proximate the dome means marked with a second angular scale, the indicator element being aligned to translate relative to the first and second angular scales by rotation of the plumb means about the two rotational axes.

16. The surgical hand tool of claim 15, further comprising:
a plumb weight disposed as a lower portion of the plumb means opposite to an upper portion of the plumb means including the indicator element, the plumb weight having a mass that is substantially larger than a mass of the upper portion of the plumb means.

17. A surgical hand tool, comprising:
a tool attachment for attaching a surgical tool oriented along a first tool axis;
a handle spanning a second tool axis;
a first dome housing defining a cut-out surface area bounded by a perimeter edge of an outer surface of said first dome housing, the perimeter edge defining an indicator edge element;
a second dome housing rotationally coupled to the first dome housing along a first rotation axis transverse to the tool axis, the second dome housing having first and second angular scales indicated on an outer surface, the first angular scale being disposed to indicate rotation of the second dome housing about the first rotation axis relative to the indicator edge element, the second angular scale being disposed along a cut-out arcuate channel surface area defined by a perimeter edge on the outer surface of said second dome housing;
a shaft disposed across a diameter at a base of the second dome housing, the shaft being oriented along a second rotation axis transverse to the first rotation axis and being oriented in a first tool plane spanned by the first tool axis and second tool axis;
a plumb body rotationally coupled to the shaft to rotate about the second rotation axis, the plumb body having a plumb marker which translates through the cut-out arcuate channel surface area to indicate rotation of the plumb body about the second rotation axis relative to the second angular scale.

18. The surgical hand tool of claim 17, further comprising:
a plumb weight disposed as a lower portion of the plumb body opposite to the plumb marker, the plumb weight having a mass that is substantially larger than a mass of the plumb marker.

19. A surgical hand tool for aligning a surgical instrument relative to at least two reference axes, comprising:
a handle means for gripping the hand tool manually;
an attachment means for receiving an elongate surgical instrument;
a first dome means having a base and an exterior surface defining an indicator means;
a second dome means having first and second angular rotation scales disposed on an exterior surface, the second dome means coupled to the first dome means to rotate about a first rotation axis, the first angular scale indicating rotation of the second dome means about the first rotation axis relative to the indicator means on the first dome means;
a plumb means fixed to a shaft disposed across a base of the second dome means, the plumb means having an indicator element disposed to indicate rotation of the plumb means relative to the second angular rotation scale on the second dome means.
